(19) United States
(12) Patent Application Publication
(10) Pub. No.: US 2006/0046982 A1
(43) Pub. Date: Mar. 2, 2006

(54) Orthomolecular medical use of L-citrulline for capillary endothelial protection and adjacent cell protection in neurodegenerative disease

(76) Inventor: William Howard Waugh, Greenville, NC (US)

Correspondence Address: LYNN E BARBER, P O BOX 16528, FORT WORTH, TX, 76162, US

(21) Appl. No.: 10/929,003

(22) Filed: Aug. 26, 2004

(51) Int. Cl. A61K 31/198; A61K 31/663

(52) U.S. Cl. 514/102; 514/565

(57) ABSTRACT

This invention embodies use of exogenous L-citrulline not only for support of capillary endothelial function, by L-citrulline bioconversion to plasma L-arginine to provide more cell substrate for enzymatic generation of nitric oxide, but also for providing more L-arginine in cellular microenvironments including that in acidified endosomes and lysosomes for potential chelation to free bivalent iron. Thus, it is contemplated that cellular oxidative damage from hydroxyl free radicals generated by Fenton-like reactions between bivalent iron and diffusible hydrogen peroxide will be ameliorated or prevented in age-related macular degeneration and in several nervous system age-related degenerative diseases by oral L-citrulline supplementation. In addition, a novel pharmaceutical composition containing L-citrulline, calcium myo-inositol hexakisphosphate (calcium phytate), and calcium carbonate is formulated in order to provide L-citrulline and also to reduce unfavorable higher serum ferritin levels to lower values by means of reduction of body iron stores, by inhibition of gastrointestinal absorption of iron without inducing substantial negative calcium balances.

### TECHNICAL FIELD

This invention relates to the field of orthomolecular medicine for the preservation of good health and the amelioration of disease by novel means of nutritional supplementation.

The invention herein provides a method, using L-citrulline not only for support of capillary endothelial function, by L-citrulline bioconversion to plasma L-arginine to provide more cell substrate for enzymatic generation of nitric oxide, but also for providing more L-arginine in cellular microenvironments including that in acidified endosomes and lysosomes for potential chelation to free bivalent iron. The invention is effective in several nervous system age-related degenerative diseases and in reduction of unfavorable higher serum ferritin levels to lower values by means of reduction of body iron stores, by inhibition of gastrointestinal absorption of iron without reducing substantial negative calcium balances. Other objects and features of the inventions will be more fully apparent from the following disclosure and appended claims.

The BACKGROUND for and the DESCRIPTION of this invention are included within the following three EXAMPLES 1, 2, and 3.

References to prior work in this field are given herein. Applicant also has two prior patents, U.S. Pat. Nos. 5,874,471 and 6,028,107. The disclosure of these patents and all other patents and publications cited herein is incorporated herein by reference.

Beneficial effects of L-citrulline supplementation are expected to occur in a number of age-related nervous system diseases associated with iron-catalyzed oxidative damage and increased local accumulation of iron and ferritin in the capillary layer of ocular choroid tissue and in nervous system capillary endothelium and its neighboring tissue. It is contemplated that the local accumulation of iron and denatured cytoskeletal macromolecules will be reduced and that cellular endosomal and lysosomal functions will be subjected to much less oxidative stress from Fenton-like reactions which generate hydroxyl free radicals that cause site-specific oxidative damage from liberated bivalent iron reacting with diffusible hydrogen peroxide.

These age-related, progressive diseases include age-related macular degeneration, Alzheimer's disease, Lewy body dementia, Parkinson's disease, progressive supranuclear palsy, multiple system atrophy, focal torsion dystonias, amyotrophic lateral sclerosis and diabetic neurodegeneration.

The beneficial effects of L-citrulline are contemplated to be mediated not only by bioconversion of orally administered L-citrulline to plasma L-arginine that increases available L-arginine as substrate in the enzymatic generation of nitric oxide for support of endothelial function and platelet stability in discrete and intricate ocular choroid and nervous system capillaries, but also by novelly replenishing or providing more available plasma L-arginine that will increase sequentially cytosolic levels of chelatable L-arginine for the oxidation-reduction chelation of free iron liberated inside active acidified endosomes and lysosomes.

Additionally, it is contemplated that oral supplementation with calcium myo-hexakisphosphate (calcium phytate) conjointly with a fourfold greater millimole proportion of calcium carbonate and with L-citrulline will novelly reduce the body stores of iron including irons stored inside vascular endothelial and neural system cells. The hazard of oxidative cell damage from Fenton-like reactions of bivalent iron and hydrogen peroxide will be reduced further.

The beneficial effect of ingested calcium myo-inositol hexakisphosphate (calcium phytate) with a fourfold greater millimole proportion of calcium carbonate is contemplated to result mainly through the mechanism of phytate binding to some luminal iron within the gastrointestinal tract, thereby resulting in less iron absorption because of this binding and obviating a positive iron balance, uncomplicated by induced substantial negative body balances of calcium, and resulting in less ferritin and less stored iron levels inside endothelial and nervous system cells. Potential Fenton-like oxidative reactions in ageing and age-related neurodegenerative diseases will be reduced novelly by this method.

The oral use of L-citrulline with (or without) calcium myo-inositol hexakisphosphate (calcium phytate) combined with calcium carbonate is contemplated to reduce neurodegenerative oxidative damage and to support normal modulation of iron and macromolecular metabolism in endothelial and nervous system cells during human ageing while maintaining or reducing serum ferritin concentrations to a preferred range between 20 and 60 microg/L rather than at higher unfavorable levels.

L-citrulline supplementation to increase available plasma L-arginine levels is contemplated to be beneficial often in diabetic neurodegenerative complications since 1) diabetes mellitus is a disease with microvascular and adjacent tissue oxidative stress (Baynes J W. Diabetes 1991; 40: 405-412) and the vascular endothelium in diabetes has a dysfunctional arginine/nitric oxide pathway which is ameliorated by the administration of L-arginine (Pieper G M and Peltier. J. Cardiovasc. Pharmacol. 1995; 25: 397-403); 2) reduced plasma arginine levels have been found both in humans with diabetes and in experimental diabetic animals (Grill V et al. Metabolism 1992; 41: 28-32; Pieper G M and Peltier B A. J. Cardiovasc. Pharmacol. 1995; 25: 397-403); and 3) serum ferritin levels and body iron stores are significantly increased in humans with diabetes and also in apparently healthy women in risk of type 2 diabetes (Wilson J G et al. AM. J. Med. Sci. 2003; 325: 332-339; Jiang R. et al. JAMA 2004; 291: 711-717).

I contemplate that endothelial dysfunction in neural capillaries as a result of oxidative damage from liberated iron stored in endothelial ferritin and cytosolic reduction of L-arginine including less L-arginine chelatable to bivalent iron in microenvironments for normal endosomal and lysosomal functions lead often to discrete diabetic central nervous system degeneration and peripheral neuropathies secondary to critical Fenton-type oxidative damage. Therefore, supplementation with L-citrulline and often conjointly with calcium phytate to reduce iron absorption is suggested as novel therapy for diabetic neurodegenerations.

### EXAMPLE 1

The 71-year old male cited in EXAMPLES 1, 2, and 6, of U.S. Pat. No. 5,874,471 continued his intake of L-citrulline capsules in two divided doses almost every day for the following 8 years, usually at 4.5 and 6.0 grams daily. Plasma L-arginine levels increased an average of 50 microMol/L two hours after ingestion of 3.0 grams of L-citrulline in capsular form following overnight fasting. At age 79, his weight was 100 kg and his body mass index calculated as 31 kg per square meter (a score of mild obesity). At the age of 78, his serum ferritin level was 26 microg/L, blood hemoglobin 13.6 g/dL. Hematocrit 38.5% and total leucocyte count was 6.800/mcL. Fasting blood glucose was within the normal range. All of the above values were determined by a commercial laboratory (Quest Diagnostics) which lists a normal serum ferritin range of 20 to 380 microg/L. At the age of 78 11/12 years, funduscopic examination by a board-certified ophthalmologic physician revealed no retinal signs of “dry” (atrophic) or “wet” (exudative) age-related macular degeneration; this included the absence of undigested abnormal deposits of lipoprotein termed drusen. No overt symptoms of Alzheimer's disease or Parkinson's disease or other dystonic disorder had developed by the age of 79 years (Beers M H and Berkow B. in The Merck Manual of Diagnosis and Therapy. 199, 1395-1399 & 1465-1470).

Serum ferritin concentrations are recognized to be very useful clinically for the estimation of the magnitude of the total body stores of iron in humans. In the art, ferritin levels below 10 or 12 microg/L indicate depletions of iron stores, while serum ferritin levels greater than 15 microg/L are proportionate generally to one's iron body stores (Facchini F S and Saylor K L. Ann. N.Y. Acad. Sci. 2002:967:342-351). Iron stores are known to increase generally with ageing.

An extreme range of normal (95% range) ferritin values for adult males of 18 to 350 and an extreme normal range for females greater than 49 years of age of 18 to 204, microg/L is reported in America (Beers M H and Berkow R. in The Merck Manual of Diagnosis and Treatment 1999; 2534). High body or local iron stores are considered risk factors for ischemic cardiovascular disease (de Valk B and Marx J J M. Arch. Intern. Med. 1999; 159: 1542-1548; Facchini F S and Saylor K L. Ann. N.Y. Acad. Sci. 2002; 967: 341-351); for age-related macular degeneration involving Fenton-like oxidative reactions (Hahn P et al. Arch. Ophthamol. 2003; 121: 1095-1105; for Alzheimer's disease (Goodman L. J. Nervous Mental Disease 1953; 117: 97-130; Connor J R et al. J. Neurosci. Res. 1992; 31; 327-335; Bishop G M et al. Dev. Neurosci. 2002; 24; 184-187); for Parkinson's disease and other neurodegenerative diseases with oxidative damage (Gutteridge J M C. Ann. Neurol. 1992; 32: S16-S21, Jenner P. Lancet 1994; 344: 796-798; Double K L et al. J. Neurochem. 1998; 70: 2492-2499; Christen Y. Am. J. Clin. Nutr. 2000; 71; 621 S-629S). None of the above authors nor Fine et al. (New Engl. J. Med 2000; 342: 483-491) suggested use of either L-citrulline or calcium myo-inositol hexakisphosphate (calcium phytate) supplementation as therapy for any of these neurodegenerative diseases which have oxidative damage.

It is claimed that oxidative damage may be early and great and that oxidative damage may become reduced with disease progression and lesion formation (Hahn P. et al. Arch. Ophthamol. 2003; 121:1095-1105; Nunomura A et al. J. Neuropath. Exp. Neurol. 2001; 60:759-767). Neurovascular injury and oxidative stress with brain capillary dysfunction may have occurred early in case 2 described by Malamud W and Lowenburg K (Arch. Neurol. Psychiat. 1929; 21: 805-827) in which Alzheimer's disease developed as a complication of scarlet fever at age of 7 with death at age of 23 11/12 years with intensely affected lesions in the choroid plexus of the brain.

I contemplate that many cases of age-related neurodegerative disease involve microvascular and capillary endothelial dysfunction with dysfunction of iron and macromolecular homeostasis that involves cytoskeletal regulation (Terry R D J. Neuropathol. E. Neurol. 1996; 55: 1023-1025) and endosomal and lysosomal disturbances with fraility and disruption of lysosomes in ageing and also in atherosclerosis (Harman D. Drugs Aging. 1993; 3:60-80; Wolinsky H. and Fowler S. New Engl. J. Med. 1978; 299: 1173-1178).

The art recognizes that the normal capillary network of the human brain is so intricate that no neuron or glial cell is no more than about 20 micrometers from a neighboring capillary (Pardrige W M Neuron 2002; 36: 555-558). I contemplate that many neurodegenerative disorders involve discrete capillary disorders in vulnerable neurons as shown in Alzheimer's disease by Scheibel A B et al. (Ann. Med. 1989; 21: 103-107), by Buee L. et al. (Acta Neuropathol. 1994; 87: 469-480), and by Claudio L (Acta Neuropathol. 1996; 91: 6-14).

It is relevant that: 1) serum ferritin levels above 50 microMol/L were found in 82% of elderly patients with Alzheimer's disease dementia and brain ferritin has been found much increased in severe Alzheimer's disease (Fischer P et al. Life Sciences 1997; 60: 2273-2278); 2) cerebrospinal fluid nitrate concentrations were significantly decreased in patients with Alzheimer's disease, Parkinson's disease, and multiple system atrophy, suggesting decreased brain production of nitric oxide by the L-arginine-nitric oxide pathway in these 3 disorders (Kuiper M A et al. J. Neurol. Sci. 1994; 121: 46-49); and 3) L-citrulline recycled for synthesis of nitric oxide in cerebral vascular endothelial cells (Lee T J F and Yu J-G. Ann. N.Y. Acad. Sci. 2002; 962: 73-80.

### EXAMPLE 2

Intracellular iron is generally considered never found uncomplexed in mammalian cells unless there is oxidative stress with Fenton-like reactions and free ionic iron is normally found only in very small concentrations of about 1 microMol/L in a transit low molecular weight cytosolic pool (Williams R J P. FSBS Letters 1982; 140: 3-10). This pool is considered to originate within endosomes after pinocytosis or within lysosomes as bivalent iron cations (ferrous) liberated from ferritin or from cellular ingredients or debris after intraendosomal or intralysosomal acidification at pHs between 5 and 6 in brain and vascular endothelial cells for subsequent extravesicular transfer (Aisen P. Ann. Neurol. 1992; 32: S62-S68; Roberts R et all. Ann. Neurol. 1992; 32: S43-S50; Wolinsky H and Fowler S. New Engl. J. Med. 1978; 299: 1173-1178).

It is well established that iron importantly catalyzes a variety of free-radical reactions that cause oxygen-dependent tissue injury (Borg D C and Schaich K M. in Oxygen Radicals and Tissue Injury. 1988, 20-26). Cyclical Fenton-like reactions of bivalent iron and diffusible hydrogen peroxide (H₂O₂) are recognized to be the driving force initiating cytotoxic free radical reactions which form site-specific, short-lived, and extremely toxic hydroxyl radicals in disease states including many neurodegenerative diseases (Fridovich I. in Oxygen Radicals and Tissue Injury. 1988; 1-5; Borg D C and Schaich K M. in Oxygen Radicals and Tissue Injury. 1988; 20-26; Jenner P. Lancet 1994; 344: 796-798).

The Fenton chemical reaction may be depicted as: Fe(11)+H₂O₂------->Fe (111)+free hydroxyl radical+hydroxide anion.

Notably, H₂O₂ is present in circulating human blood plasma at normal levels of about 2.5 microMol/L or more to higher abnormal levels even over 55 microMol/L. (Varma S D and Devanmanoharan P S. Free Rad. Res. Comms. 1991; 14: 125-131; Smielecki J et al. Intl. J. Cardiol. 1996; 55: 137-143; Lacy F et al. J. Hypertens. 1998; 16: 291-303).

The interaction of bivalent iron and H₂O₂ in causing oxidative denaturation and insolubility of glycoprotein components synergistically by Fenton-like reactions is demonstrated in the following EXAMPLE TESTS at acidity approximating that in activated brain endosomes (pH 5.6) during cell iron homeostasis (Aisen P. Ann. Neurol. 1992; 32: S62-S68).

Human plasma Cohn fraction 1V-1 protein solutions (predominately alpha glycoglobulins, rich in lipid—obtained from Sigma Chemical Co.) of 40 mg/dl in saline solutions (140 NaCl mMol/L) were employed at existing pHs of 5.7-5.8 without additional confounding non-protein buffer. A nephelometer measured the intensity of reflected Tydall light. Developed turbidities were measured to quantify the insolubities of the dissolved proteins that were induced by introducing 100 microMol/L of ferrous sulfate and 40 microMol/L of H₂O₂ with incubations at 37° Celsius for 30 minutes. Insolubilities were standardized as the percent of maximal possible induced turbidities by comparison to similar protein solutions after mixing with the protein-precipitating agent sulfosalicylic acid at 1.5 g/dL.

Twelve control turbimetric tests averaged 7.28±0.33% (mean & standard error) with combined use of the ferrous salt and H₂O₂. In 4 separate tests each, addition of L-arginine free base to 300 and 400 microMol/L before addition of the ferrous salt and H₂O₂ resulted in mean inhibition by 91.7% and by 100.0% (complete), respectively, of the insolubities induced by the Fenton-like reactions. The induced mean turbidities were only 0.56±0.29% and 0.00±0.00%, respectively. Use of 40 microMol/L H₂O₂ in the absence of the ferrous salt failed to cause turbidity. Three other amino acids tested at 400 microMol/L (glycine, L-glutamine, and L-citrulline) did not inhibit these Fenton-like reactions. L-histidine free base, another dibasic amino acid, at 400 microMol/L inhibited the denaturations by only a mean of 52.6%, indicating about half as much inhibition compared to the complete prevention by arginine at 400 microMol/L. It is known in the art that L-arginine is a stronger dibasic amino acid than L-histidine with a much greater third dissociation constant (pK3) and therefore L-arginine is much more protonated at pH's of 5.7-5.8 for oxidation chelation of iron.

Visible yellow hues developed in the reaction solutions rapidly after mixing the L-arginine with the ferrous salt before final solution addition of the H₂O₂. This xanthochromia indicated a change of bivalent iron to trivalent iron at pH of 5.7-5.8, caused by ready charge transfer to the available L-arginine as ligand, by coordinate complexing in oxidation-reduction chelation with iron change to its typical trivalent yellow color (Miessler G I and Tarr D A. In Inorganic Chemistry 2004; 197-198 and 440-446). Thus, it is shown novelly that L-arginine is an outstanding chelatable amino acid of iron in acidity similar to that in activated endosomes and lysosomes.

It is recognized in the art that intracellular pools of L-arginine might become depleted from their usual endothelial levels of about 2 to 4 milliMol/L during periods of prolonged release of nitric oxide as endothelial-derived relaxing factor (Hecker M et al. Proc Natl. Acad. Sci. U.S.A. 1990; 87: 8612-8616). I contemplate that with ageing cytosolic insufficiency of chelatable L-arginine develops at times with abnormally reduced L-arginine concentrations in endothelial and neural cells in discrete and select parts of the choroid vasculature in age-related macular degeneration and in discrete and select parts of the central nervous system in age-related nervous system diseases.

It has been shown that L-arginine—derived nitric oxide, containing an unpaired electron, can by itself act as an antioxidant as unpaired electron, can by itself act as an antioxidant by direct chelation as ligand to bivalent iron to inhibit or prevent Fenton-like oxidative reactions at pHs of 7.0-7.3 in the presence of micromole/L concentrations of hydrogen peroxide (Kanner L et al. Arch. Biochem. Biophysics 1991; 289: 130-136).

This cytosolic chelatable arginine insufficiency is contemplated to occur in a variety of progressive neurodegenerative diseases, particularly if plasma levels of arginine decline with ageing as has been reported (Moriguti J C et al. Amino Acids. 1995; 9: 46-47) or when the plasma levels of competitive asymmetrical dimethyl L-arginine (ADMA) increase with ageing sufficiently to inhibit the normal physiologic functions of L-arginine in vascular endothelial cells, as was shown recently (Leiper J and Vallance P. Cardiovasc. Res. 1999; 43: 542-548; Bode-Boger S M et al. Vascular Med. 2003; 8: 77-81).

Thus, I contemplate that age-related insufficiency of L-arginine leads to endothelial dysfunction with impaired iron homeostasis and oxidative stress consequent to functional impairment of endosomes and lysosomes. Furthermore, I contemplate that several age-related neurodegenerative diseases will be ameliorated or preventable in part by increased plasma and extracellular levels of L-arginine made available by the novel means of orally ingested L-citrulline as precursor agent. The art has shown that orally given L-arginine improved endothelial function in healthy individuals older than 70 years, probably due to normalization of L-arginine/ADMA ratios (Bode-Boger S M et al. Vascular Med. 2003; 8: 77-81).

### Dietary Formulation

To the extent that serum ferritin levels of adult subjects are unduly high, for example above 50 microg/L, a preferred embodiment of this invention uses a noninvasive method to reduce serum ferritin levels and reduce body stores of iron to near depletion of stores. This method is in contrast to the use of quantitative bloodlettings to reduce iron stores and the risk of atherosclerosis in high-risk individuals with iron stores within normal limits and serum ferritin levels within normal limits but with relatively high ferritin levels which averaged 272 microg/L (Facchini F S and Saylor K L. Ann. N.Y. Acad. Sci. 2002; 967: 342-351).

The embodiment entails the oral intake daily of a moderate amount of a natural dietary substance, myo-inositol hexakisphosphate (phytate), which is found in edible legumes, cereal grains, and nuts (Graf E et al. J. Biol. Chen. 1987; 262: 11647-11650; Shamsuddin AM. J. Nur. 1995; 125; 725S-732S); Hendler S S, chief editor. in PDR for Nutritional Supplements. 2001; 1^{st} edition: p. 222-223). This embodiment employs the monocalcium salt of myo-inositol hexakisphosphate (calcium phytate) plus concurrent use of calcium carbonate to counteract in the lumen of the gastrointestinal tract some of the intrinsic acidity of calcium phytate. Phytate has a unique structure with 6 dihydrogen phosphate groups and the monocalcium chelated phytate leaves 10 of these hydrogens in the phytate not “neutralized” by counter-cations.

Calcium phytate has a formula weight of 698.1 and a CAS No. of 7776-28-5. The compound is commercially available and the powdered form may be obtained in purified, bulk quantities from Sechuan Jempai Co. Sichuan, China, P.C. 610051. Alternatingly, phytate may be separated from a natural source, commonly from corn or rice, by known chemical means and then converted to the insoluble monocalcium salt.

Formed diferric and tetraferric phytate complexes are insoluble and are not absorbed from mucosal cells of the gastrointestinal tract (Skikne B and Baynes R D in Iron Metabolism in Health and Disease, Brock, J H et al. editors, 1994, p. 162). Monoferric chelation to phytate is recognized to block hydroxyl free radical formation. (Graf E et al. J. Biol. Chem. 1987; 262: 11647-11650).

It has been shown that large dietary intakes of about 2.5 grams daily of phytate (mainly in the form of 4.5 grams of dodecasodium phytate hydrate) can lead to negative body balances of calcium while reducing serum iron and calcium levels (Reinhold J G et al. Lancet 1973; 1: 283-288). It is known that at pH of 7.4, phytate forms complexes with divalent metal ions in decreasing order of: copper>zinc>iron>calcium (Shamsuddin AM. J. Nutr. 1995; 125: 725S-732S).

Therefore, I formulate a method that entails use of calcium phytate combined with concurrent administration of calcium carbonate (precipitated. U.S.P.) in a fourfold greater millimole proportion than the employed calcium phytate, which lessens the potential acidity induced by luminal calcium phytate. An additional beneficial effect of this recommended dietary formulation containing L-citrulline, calcium phytate, and calcium carbonate is that the contained ingested phytate may lower total serum cholesterol and triglyceride levels in humans by yet undetermined means, as had been demonstrated in rats with dietary use of monopotassium phytate (Jariwalla R J et al. J. Applied Nutr. 1990; 42: 18-28).

The following is the formulation, although other three ingredient pharmaceutical compositions including L-citrulline and calcium phytate as two of the active ingredients may be used by one experienced in the art. A convenient name for the formulation is CitCalPhyt.

### EXAMPLE 3

| | |
|---|---|
| CAPSULES | 500 mg L-Citrulline |
| | 100 mg Calcium Phytate |
| | 58 mg Calcium Carbonate |
| L-Citrulline | 5.00 g |
| Calcium Phytate | 1.00 g |
| Calcium Carbonate, USP | 0.58 g |

Mix the above three ingredients by trituration in a large mortar and pestle or blend in a blender with tumble blending for a few minutes. Then, fill No. 00-sized hard gelatin capsules with mild pressure to an average weight of about 658 mg per blend per capsule. Capsules are stored in a cool, dry place. Two or three capsules per serving are taken two or more times daily as necessary to lower unduly high serum ferritin levels, coupled with the included supply of L-citrulline as precursor amino acid for bioconversion to L-arginine.

While the invention has been described with reference to specific embodiments, it will be appreciated that numerous variations, modifications, and embodiments are possible, and accordingly, all such variations, modifications, and embodiments are to be regarded as being within the spirit and scope of the invention.

What is claimed is:

1. A method of improving the health of a subject who has a risk factor for age-related progressive neurodegenerative disease, to increase the plasma level of arginine in the subject to a greater level, comprising orally administering L-citrulline, calcium phytate and calcium carbonate in an amount sufficient to reduce acidity induced by calcium phytate and to maintain serum ferritin concentrations at low normal values, for support of capillary endothelial functions and for increased L-arginine as cytosolic ligand for reduction in oxidative damage from divalent iron in acidified microenvironments within endosomes, lysosomes, and phagosomes.

2. The method according to claim 1, wherein the subject has not manifested an apparent ocular choroid or brain progressive neurodegenerative disease.

3. The method according to claim 1, wherein the subject has age-related macular degeneration.

4. The method according to claim 1, wherein the subject has Alzheimer's disease.

5. The method according to claim 1, wherein the subject has Lewy body dementia.

6. The method according to claim 1, wherein the subject has Parkinson's disease.

7. The method according to claim 1, wherein the subject has progressive supranuclear palsy.

8. The method according to claim 1, wherein the subject has vascular dementia including subcortical white matter disease.

9. The method according to claim 1, wherein the subject has a focal torsion dystonia.

10. The method according to claim 1, wherein the subject has amyotrophic lateral sclerosis.

11. The method according to claim 1, wherein the subject has multiple system atrophy.

12. The method according to claim, 1 wherein the subject has diabetic neuropathy.

13. The method according to claim 1, wherein L-citrulline is administered for the support of the health condition in a dosage range of from about 1.0 to about 15 grams per day.

14. The method according to claim 1, wherein the L-citrulline is administered in a form selected from the group of: capsule, tablet, and liquid.

15. The method according to claim 1, wherein the L-citrulline is administered at least twice daily.

16. The method according to claim 1, wherein the L-citrulline, calcium phytate and calcium carbonate are administered at least twice daily.

17. The method according to claim 1, wherein the L-citrulline, calcium phytate and calcium carbonate are administered in a form selected from the group of: capsule, tablet, and liquid.

18. A method of improving the health of a subject who has a risk factor for age-related progressive neurodegenerative disease, to increase the plasma level of arginine in the subject to a greater level, comprising orally administering L-citrulline for support of capillary endothelial functions and for increased L-arginine as cytosolic ligand for reduction in oxidative damage from divalent iron in acidified microenvironments within endosomes, lysosomes, and phagosomes.

19. A composition for improving the health of a subject who has a risk factor for age-related progressive neurodegenerative disease, to increase the plasma level of arginine in the subject to a greater level, comprising L-citrulline, calcium phytate and calcium carbonate in an amount sufficient to reduce acidity induced by calcium phytate and to maintain serum ferritin concentrations at low normal values.
